(19) United States
(12) Patent Application Publication
(10) Pub. No.: US 2005/0085699 A1
(43) Pub. Date: Apr. 21, 2005

(54) Attachment devices for surgical instruments

(76) Inventor: Sol Weiss, Reseda, CA (US)

Correspondence Address: Mitchell, Silberberg & Knupp, LLP, 11377 West Olympic Boulevard, Los Angeles, CA, 90064, US

(21) Appl. No.: 10/904,998

(22) Filed: Dec. 8, 2004

Related U.S. Application Data
(63) Continuation-in-part of application No. 10/095,950, filed on Mar. 11, 2002, now Pat. No. 6,830,547.
(63) Continuation-in-part of application No. 10/122,450, filed on Apr. 13, 2002.
Application No. 10/095,950 is a continuation-in-part of application No. 09/978,604, filed on Oct. 15, 2001, now Pat. No. 6,394,950.
(60) Provisional application No. 60/241,067, filed on Oct. 17, 2000.
(60) Provisional application No. 60/286,188, filed on Apr. 24, 2001.
(60) Provisional application No. 60/286,750, filed on Apr. 26, 2001.

(51) Int. Cl. A61B 1/32

(52) U.S. Cl. 600/221

(57) ABSTRACT

Provided is an apparatus for insertion into a port of a speculum. The apparatus has an insertion member that includes one or more sloped elements on (e.g., protruding from) its outer surface, for creating a secure fit (e.g., a friction fit or a snap fit) with the inner surface of the port. In certain representative embodiments, the sloped element(s) slope significantly more gradually from the distal end of the male insertion member than from its proximal end, and more preferably have a sudden drop off near their proximal ends.

This application is a continuation-in-part of application Ser. No. 10/095,950 filed Mar. 11, 2002 (the '950 application) and of application Ser. No. 10/122,450 filed Apr. 13, 2002 (the '450 application). The '950 application is a continuation-in-part of application Ser. No. 09/978,604 filed Oct. 15, 2001, now U.S. Pat. No. 6,394,950 (the '950 patent), which issued May 28, 2002, and which claims the benefit of Provisional Application No. 60/241,067 filed Oct. 17, 2000 (the '067 application), and Provisional Application No. 60/286,188 filed Apr. 24, 2001 (the '188 application). The '450 application claims the benefit of Provisional Application No. 60/286,750 filed Apr. 26, 2001 (the '750 application). Each of the foregoing patents and patent applications is incorporated by reference herein as though set forth herein in full.

### FIELD OF THE INVENTION

The present invention pertains to attachment devices for attaching one surgical instrument to another, e.g., for use in abdominal, thoracic, vaginal and other body procedures. Such attachment devices are particularly useful for attaching surgical instruments to a speculum or similar device.

### DESCRIPTION OF THE RELATED ART

Certain surgical, diagnostic and other devices are used for examining cavities and surgical openings that may include prior art devices known as speculums, retractors, or other investigative instruments that require an attachable device to allow for holding a light source, additional instrumentations, suctioning equipment and irrigation tubes without obstructing the view of the operator or the surgeon.

The '188 application, discloses a light cone for use with a surgical instrument, such as the speculum in the present inventor's U.S. Pat. No. 5,868,668, which is incorporated by reference herein, and/or any of the speculums disclosed in any of the other patents and patent applications referenced above.

In the present inventor's earlier patents, it was shown how the cone of light served both for transmission of light and for providing a fairly tight fitting when inserted into a receptacle. However, the present inventor has continued to improve designs in connection with attachment devices for surgical instruments, resulting in the present invention.

### SUMMARY OF THE INVENTION

One result of these efforts is the provision of an apparatus for insertion into a port in a speculum. The apparatus has an insertion member that includes one or more sloped elements on (e.g., protruding from) its outer surface, for creating a secure fit (e.g., a friction fit or a snap fit) with the inner surface of the port. Preferably, the sloped element(s) slope significantly more gradually from the distal end of the male insertion member than from its proximal end, and more preferably have a sharp drop off near their proximal ends.

By virtue of the foregoing arrangement, any possibility of the attachment apparatus inadvertently coming out of the speculum port during a surgical procedure often can be eliminated. In fact, the attachment apparatus can be configured so as to make it very difficult or even nearly impossible to remove the attachment apparatus from the speculum.

In more particular aspects, the insertion member may function as a guide member or a guiding means for guiding, for example: light, airflow, fluid or a surgical tool or instrument. When guiding light, it is preferable that a distal end of the guide member (or guiding means, as applicable) from which the light emerges is cut at an angle so as to bend the light toward the center of the speculum. Alternatively, the guide member may be configured as (or to include) a hollow tube, e.g., for directly guiding fluids and/or air or for guiding a surgical instrument.

Preferably, the attachment apparatus also is provided with attachment means coupled to the insertion member for accepting or attaching to a surgical tool. For example, such an attachment means may be configured as (or to include) a tube-like structure for inserting a surgical tool and/or for fitting a flexible tubing around.

The male insertion member preferably: is comprised of plastic; has a non-circular cross section that matches the non-circular cross section of the port of the speculum, thereby preventing inadvertent rotation of the male insertion member in the port; and/or has at least two sloped elements on its outer surface (e.g., distributed at least approximately uniformly around the circumference of the male insertion member), each sloping upwardly from the distal end of the male insertion member toward its proximal end.

Another result of the inventor's efforts is the provision of an apparatus for attaching to the upper blade of a speculum. The apparatus includes: an upper member; a lower member disposed beneath the upper member; a guide member disposed beneath the lower member for guiding at least one of light, airflow, fluid or a surgical instrument; and a notch attached to at least one of the upper member or the lower member. The notch either is provided on the upper member and extends toward the lower member or is provided on the lower member and extends toward the upper member. In either event, with the upper member and the lower member sandwiching an upper blade of a speculum, the notch engages an outer edge of the upper blade of the speculum.

Preferably, the lower member is at least approximately parallel to the upper member; the notch is disposed on the upper member; the lower member is separated from the upper member by a distance that is just large enough to accommodate the width of the upper blade of the speculum; the entire apparatus is formed from a flexible plastic material; the notch is disposed at an outer distal corner of at least one of the upper member or the lower member; the notch is triangular shaped; the guide member is for guiding light; a distal end of the guide member from which the light emerges is cut at an angle so as to bend the light toward the center of the speculum; both the upper member and the lower member are substantially flat; the apparatus further comprises a tube-like structure coupled to the guide member for accepting a surgical tool; the apparatus further comprises attachment means coupled to the guide member for at least one of accepting or attaching to a surgical tool; the engagement of the notch against the outer edge of the upper blade of the speculum resists a tendency of the apparatus to slide off of the speculum; and/or the guide member comprises a hollow tube.

According to another aspect of the invention, an apparatus is provided for attaching to the upper blade of a speculum. The apparatus includes: a slotted member having an upper member and a lower member that together form a slot for accepting an upper blade of a speculum; guiding means disposed beneath the lower member for guiding at least one of: light, airflow, fluid or a surgical instrument; and a notch attached to at least one of the upper member or the lower member and directed inwardly into the slot. With the slotted member installed on the upper blade of the speculum, the notch engages an outer edge of the upper blade of the speculum.

Preferably, the lower member is at least approximately parallel to the upper member; the notch is disposed on the upper member; the lower member is separated from the upper member by a distance that is just large enough to accommodate the width of the upper blade of the speculum; the entire apparatus is formed from a flexible plastic material; the notch is disposed at an outer distal corner of at least one of the upper member or the lower member; the notch is triangular shaped; the guiding means is for guiding light; a distal end of the guiding means from which the light emerges is cut at an angle so as to bend the light toward the center of the speculum; both the upper member and the lower member are substantially flat; the apparatus further comprises attachment means coupled to the guiding means for at least one of accepting or attaching to a surgical tool; the engagement of the notch against the outer edge of the upper blade of the speculum resists a tendency of the apparatus to slide off of the speculum; and/or the guiding means comprises a hollow tube.

The foregoing summary is intended merely to provide a brief description of the general nature of the invention. A more complete understanding of the invention can be obtained by referring to the claims and the following detailed description of the preferred embodiments in connection with the accompanying figures.

### BRIEF DESCRIPTION OF THE DRAWINGS

FIG. 1 is an angled view of a speculum (refer to cross reference to related application).

FIG. 2 is a rear view of the speculum shown in FIG. 1 (refer to cross reference to related application).

FIG. 3 is an elevational view of a light transmitting device adapted to be used with the device of FIGS. 1 and 2.

FIG. 4 is a detailed view showing the tapered distal end of a light-carrying cone;

FIG. 5 is a top plan view of the device of FIG. 3;

FIG. 6 is a view taken along lines 13-13 of FIG. 3;

FIG. 7 is an elevational view of a prior art illuminating device used with the device of FIG. 3;

FIG. 8 is an elevational view of a resilient sleeve adapted to be used with the device of FIG. 3; and

FIG. 9 is a view taken along lines 9-9 of FIG. 7

FIG. 10 is a view of both the light-transmitting device with the attachment plate, 202, and the speculum blades (upper and lower).

FIG. 11 is a presentation of the light-transmitting device fitting tightly onto the speculum's upper blade by means of the attachment plate.

FIG. 12 is a view of the attachment plate to be used in a similar manner as shown in FIG. 11 for receiving an adapted conforming hook placement in the recess, 204.

FIG. 13 is a view of the attachment plate and a hollow tube to be used for passing suction tubes and other instruments into the operating field.

FIG. 14 is a perspective view of a speculum and an insertion apparatus therefor.

FIG. 15 is a top cross-sectional view, taken along cut line 15-15 of FIG. 14, showing how the insertion apparatus is inserted into the speculum.

FIG. 16 is a rear elevational view of the speculum shown in FIG. 14, with the insertion apparatus attached.

FIG. 17 is a left perspective view of a speculum with two attachment apparatuses attached to it.

FIG. 18 is a right perspective view of the speculum and two attachment apparatuses that are shown in FIG. 17.

FIG. 19 is a top perspective view of a light-guiding attachment apparatus for attaching to a speculum.

FIG. 20 is a right-side elevational view of the apparatus shown in FIG. 19.

FIG. 21 is a close-up view of the distal end of the attachment apparatus as shown in FIG. 20.

FIG. 22 is a bottom perspective view of the apparatus shown in FIG. 19.

FIG. 23 is a top plan view of a suctioning attachment apparatus for attachment to a speculum.

FIG. 24 is a left-side cross-sectional view of the apparatus shown in FIG. 23.

FIG. 25 is a bottom perspective view of the apparatus shown in FIG. 23.

FIG. 26 is a top perspective view of the apparatus shown in FIG. 23.

FIG. 27 is a perspective view of an attachment apparatus having hooks of varying heights, for attachment to a speculum.

FIG. 28 is a front elevational view of the attachment apparatus shown in FIG. 27.

FIG. 29 is a left-side elevational view of the attachment apparatus shown in FIG. 27.

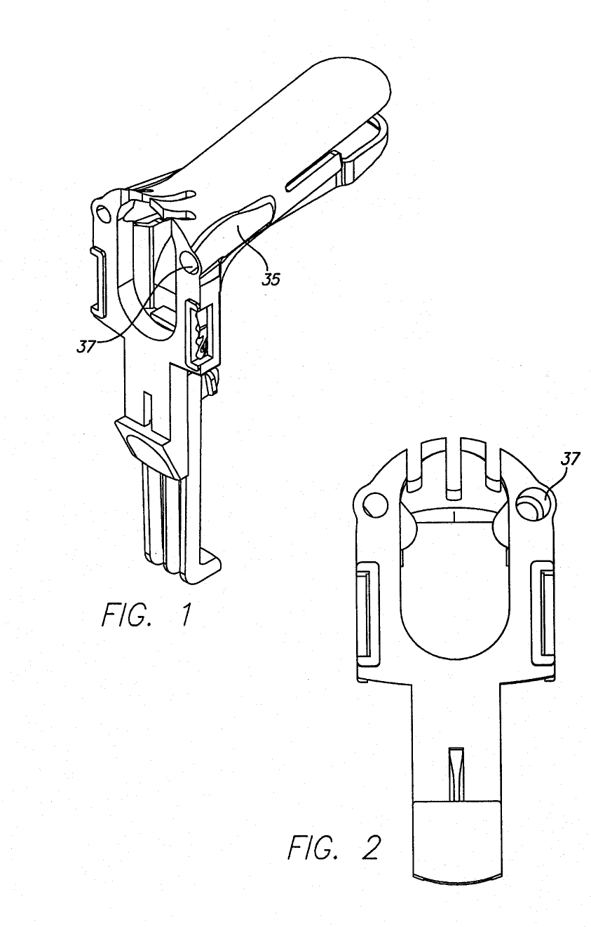

### DESCRIPTION OF THE PREFERRED EMBODIMENT(S)

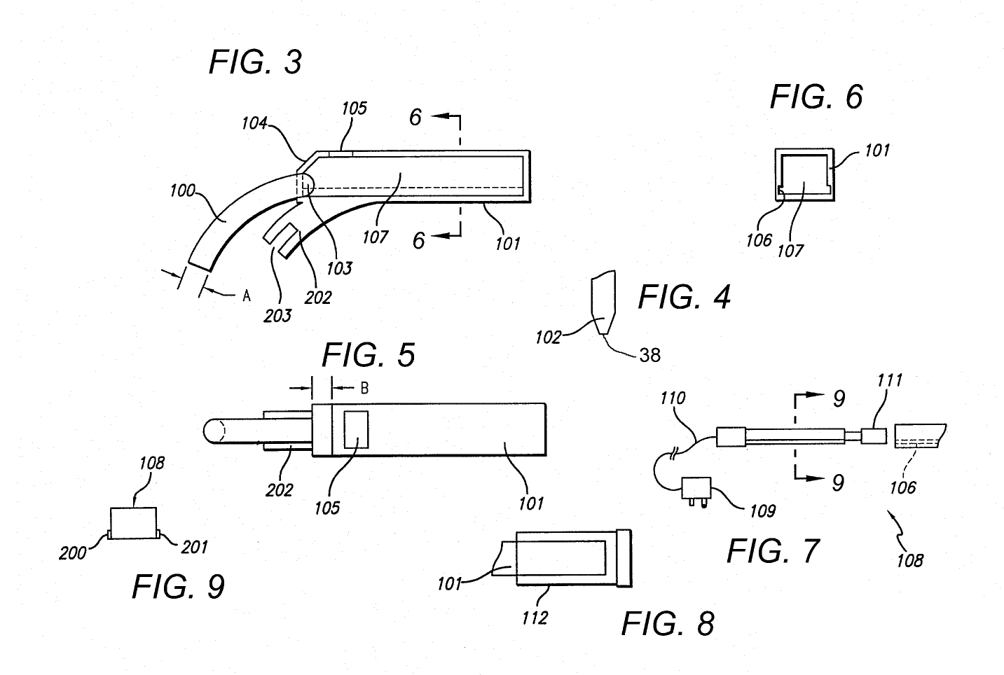

As seen in FIG. 3, a portable light carrying cone 100 is shown having a base or connector body 101, which may be of clear plastic, directing light from an illuminator or other light sources. The cone 100 shown here is solid and has a tapered distal portion 102 (FIG. 4) and a bulblike proximal end 103 (FIG. 3), within the connector body 101. The tapered portion of 102 of the cone 100 is used to fit snugly or tightly into the end 37 (FIG. 1) of cone 35 so that light can be transmitted. The bulb shaped proximal portion 103 (FIG. 3) acts as a magnifier to attract light rays concentrating them into the cone 100 for transmitting the light. The tapered portion 104 of the connector body 101 allows for non-cone absorbing light to be scattered into the area where the cone 100 and body 101 of the connector are directed.

In the '950 patent, the attachment relied upon a tapered cone fitting snugly into a tapered receptacle 37 of a speculum. The '950 patent discloses in addition an attachment process 202 wherein the tapered cut out that may be curved or straight, 203, is attached to the rim of speculums or edge of retractors, other diagnostic or surgical devices and other instrumentations that would allow for the light cone to transmit light to the desired location.

This attachment plate may also lend to holding not only cones of light but other instruments. These adaptations would permit instruments to be attached and held in a position which would not obscure the vision of the surgeon and/or operator. They may be of metal, plastic or combinations thereof.

These portable attachment devices can be metal or plastic. As a plastic device, it can be disposable so as to avoid sterilization procedures. This provides an economic advantage saving time and costs.

FIGS. 3 and 5 show the window opening 105 in body 101 that allows for heat to be dissipated, and the tracks 106 at the bottom of the opening 107 stabilize the illuminator body 111 and center it so that the bulb of the lighting device, to be discussed, does not come in contact with the walls of the connector body 101.

FIG. 7 shows a conventional prior art illuminating device 108 having a transformer 109, an electrical cord 110, and a light source 111. Light source 111 is shown inserted into cone 100, the terminal or bulb and extending to bulb end 103 (FIG. 3). Any suitable illuminator may be used. For example, the vaginal illuminator system No. 78103 manufactured and sold by Welch Allyn of Skaneateles Fall, N.Y. may be used. The opening 107 and sidewalls (FIG. 6) of housing 101 accommodate the outer configuration of light source 111.

The angled tapered front portion 104 of the body 101 permits light to be dispersed to different areas that are targeted as well. The body 101 of the connector also has tracks or slots 106 (FIG. 6) to receive flanges 200, 201 on the outer body of the illuminator 111 and its bulb to allow for not stabilizing the light bulb thereof into the center of the cavity 107 of the body 101 so that is does not contact the walls (FIG. 6) of the connector body 101.

The posterior portion of the connector body 101 may have an elastic or rubber adapter 112 (FIG. 8) with a hollow tubular interior so that the end of the body 101 fits therein for attaching to different light illuminating devices which may be of different sizes.

The cone 100 and connector body 101 are portable, and they are not dependent on other devices or handles. They are sacrificed with each procedure, as other equipment requires them to be.

The cone 100 and body 101 may be of a simplified plastic construction that allows for easy manufacturing as a two-part injection mold for plastic applications. This can also be made by metal molding or casting. The tapered tip 102 produces a tight connection with a circular opening, such as opening 37, in any instrument receptacle designed for this procedure.

Body 101 has an open area 105 of the connector body 101 near the cone projection that allows heat to escape caused by the bulb of illuminator 111.

Cone 100 has a curved cone projection 103 that may be solid plastic and provides the transmission of the light from the bulb of illuminator 111 or any other source to a specific area directed by the tip of the cone 100 out through end 38 which my be of clear plastic.

There thus is disclosed in FIGS. 3 to 8 a light connector possessing at its front portion a curved solid plastic cone tapered at its distal end for light transmission. The connector body unites with the cone having a bulb-shaped proximal portion that allows for light to be collected and transmitted to the end of the curved cone. This allows light to be carried into difficult areas as well as directed to the front of medical devices through an aperture that permits the tapered cone to fit tightly. The connector body of this unit has a slot opening in the rear portion permitting the light bulb and the illuminator body to penetrate into the cavity of the connector body. The connector body has a tracking mechanism that keeps the illuminator and light bulb in a central position without touching the inner walls of the plastic housing of the connector body. The front portion of the connector body has a window opening that permits the escape of heat from the illuminator bulb.

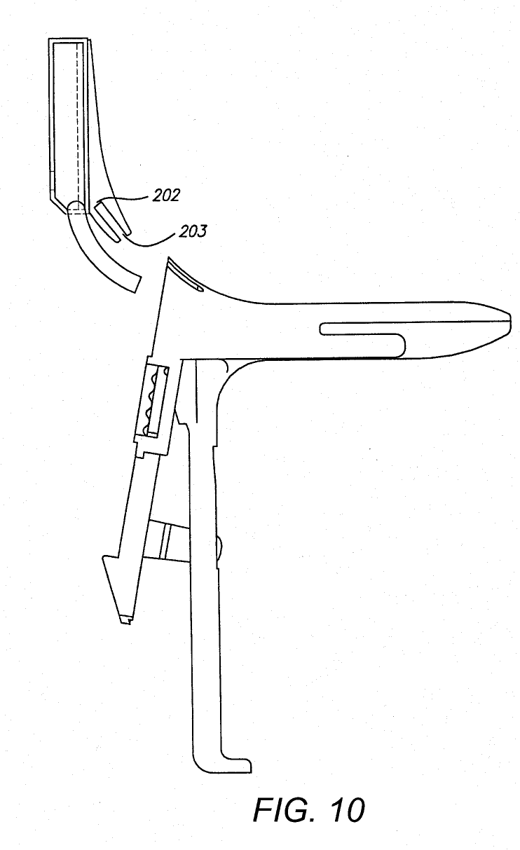

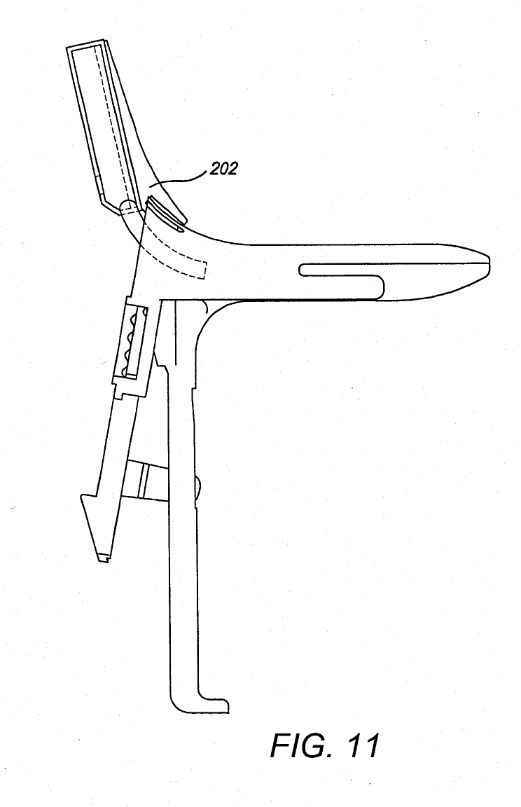

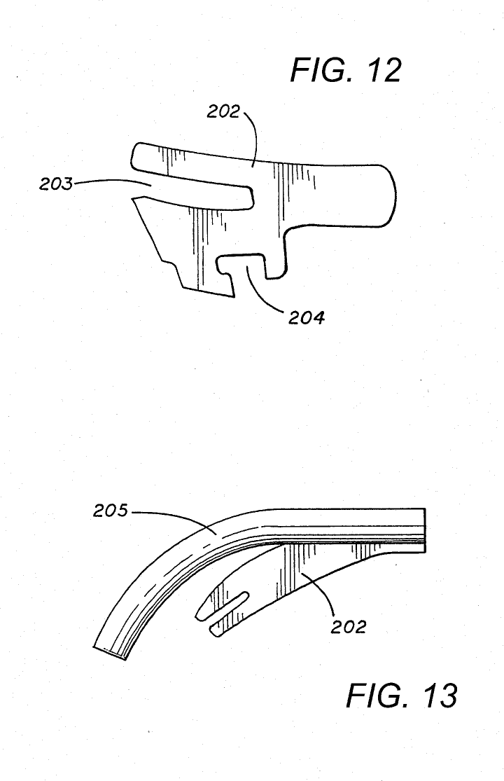

There is disclosed in FIGS. 3, 5, 10, 11, 12 and 13 an attachment plate 202 having a notch 203, respectively, that allows important functions. First, it permits the cone of light, FIGS. 3 and 5, to be attached to speculums as seen in FIGS. 10 and 11, Secondly, attachments of devices can occur with retractors and other instrumentations where it is advantageous to conduct operations without the use of assistants while holding these devices in place. This attachment plate 202 also allows a receptor recess 204 in FIG. 12 for instruments with hooks to be held in place. A third application for the attachment plate comprises a tube addition 205 permitting tube insertions for suctioning and variable size instrumentations for other operations.

Insertion Device with Sloped Protruding Elements

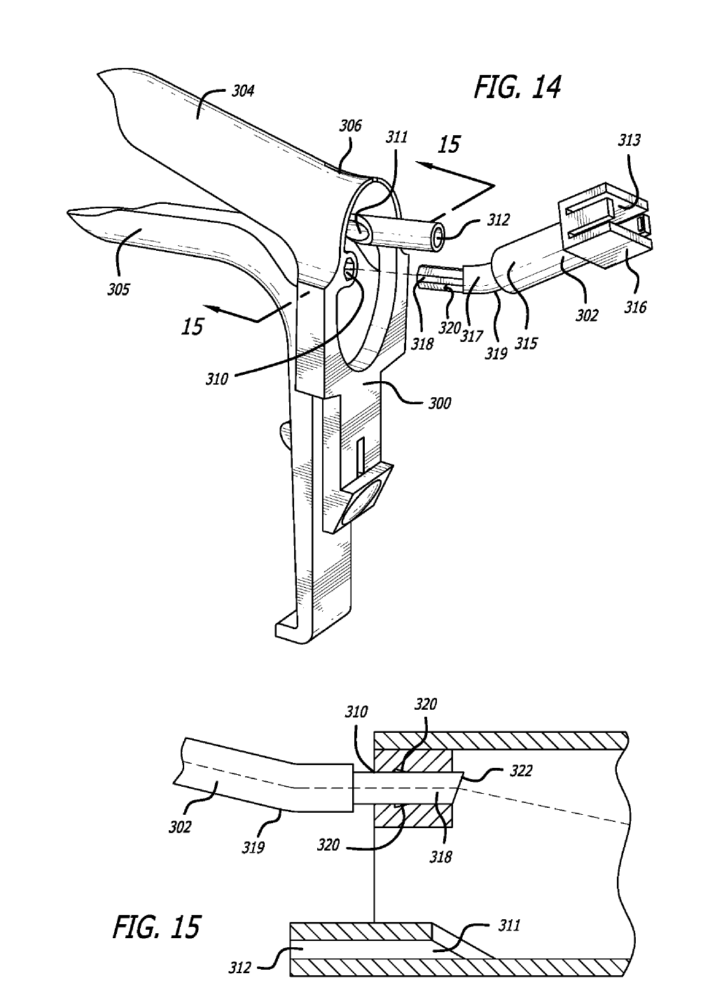

FIG. 14 shows a perspective view of a speculum 300 and an insertion apparatus 302 therefor. In the present embodiment of the invention, speculum 300 is made entirely of clear plastic. However, it instead may be made of any other material or combination of materials. As shown, speculum 300 has an upper blade 304 and a lower blade 305. A slot 306 is provided in upper blade 304, which may be used for a purpose that is described in more detail below. In addition, speculum 300 has an attachment port 310 near the bottom left corner of upper blade 304 and an attachment port 312 near the bottom right corner of upper blade 304.

In the current embodiment, port 310 is at least approximately flush with the rest of the structure of speculum 300, while port 312 protrudes rearwardly from the rest of the structure of speculum 300. Such rearward projection permits a tube (e.g., a suction tube) to be fitted onto the outside surface of port 312, and then fluids may be sucked through the interior opening 311 of port 312.

On the other hand, port 310 primarily is configured for permitting an attachment device to be inserted therein. An example of such an insertion device is apparatus 302. As shown, insertion device 302 is provided with an attachment structure 316 at its proximal end and an insertion member 318 at its distal end.

In the present embodiment, attachment structure 316 is a tube-like structure. It is elongated and has a hollow interior, open at one end, permitting a light source or other device to be inserted therein. As shown, the preferred tube-like structure is comprised of two parts: the very proximal end 313 being rectangular in cross-section and the more distal portion 315 being circular and slightly narrower in cross-section. One example of a light source that may be used in accordance with attachment device 302 is the vaginal illuminator system No. 78103 manufactured and sold by Welch Allyn of Skaneateles Falls, N.Y., as mentioned above. However, any other attachment structure instead may be used. For example, attachment structure 316 may be configured primarily for attaching a tube around its outer diameter (similar to port 312) or may be provided with a threaded, snap-fit or any other coupling mechanism.

In the present embodiment, insertion member 318 also functions as a light guide. More preferably, it is an elongated structure made from solid clear plastic, or some other optically transmissive medium. However, any other structure for guiding light instead may be used. Alternatively, insertion member 318 may be configured for guiding air, fluids or any of a variety of surgical instruments (e.g., a fiber-optic cable or a suctioning hose). In these alternate embodiments, insertion member 318 may be configured as a hollow tube, in which case air and/or fluids may be drawn directly through such a hollow tube or the hollow tube may be used to guide a suctioning tube or other surgical instrument.

Both insertion member 318 and port 310 preferably have a non-circular shape, so that attachment apparatus 302 will not rotate when inserted into port 310. More preferably, that shape is polygonal (here, hexagonal) so that apparatus 302 may be inserted into port 310 at any of a variety of (here, 6) different rotational positions. It is noted that in the present embodiment, the light guide is comprised of both insertion member 318 and a solid clear plastic portion 317 having a circular cross-section and an elbow bend 319 for bending the light beam from an angle that is more convenient for inserting a light source into attachment apparatus 302 to a direction that is more convenient for use. It is noted that a similar elbow bend may be utilized when guiding air, fluid or a surgical instrument.

In any event, the main structure of insertion member 318 preferably just fits within port 310. However, even with a fairly close fit, the present inventor has discovered that without making some additional accommodation, attachment apparatus 302 often will tend to fall out of port 310 when in use. Accordingly, insertion member 318 preferably is provided with one or more sloped elements 320 that extend from its outer surface, each sloping upwardly from the distal end of the male insertion member toward its proximal end.

As shown in FIG. 15, in the present embodiment two such sloped elements 320 are provided, exactly opposite each other around the circumference of insertion member 318. However, any other number of sloped elements 320 instead may be provided.

In the preferred embodiments, elements 320 are arranged symmetrically around the circumference of insertion member 318, or at least uniformly around the circumference of the insertion member 318 (without necessarily being at the same position along its length), in order to provide fairly uniform resistance to accidental dislodgment.

In the current embodiment, as shown in FIG. 15, the longitudinal cross-sections of elements 320 preferably are at least approximately triangular. However, a more curved surface instead may be used. Because elements 320 preferably are formed of plastic or another flexible material, they deform as insertion member 318 is inserted into port 310, thereby creating a frictional compression fit with the inner surface of port 310. Moreover, the sloped configuration of elements 320 allows insertion member 318 to be inserted into port 310 with significantly less effort than attempting to withdraw insertion member 318 from port 310. This effect can be enhanced, as shown in the drawings, by utilizing a more gradual slope from the distal end of insertion element 318 to the peak of the sloped element 320 than from such peak to the proximal end. More preferably, a very steep (e.g., perpendicular in the present embodiment) drop off is used.

In fact, in certain embodiments of the invention, it often will be extremely difficult to remove insertion member 318 from port 310 once it has been inserted. Accordingly, particularly if speculum 300 and insertion apparatus 302 are both made of plastic, the entire assembly typically can be discarded after a single use. By making both components disposable (e.g., entirely of plastic), a significant amount of cleaning and disinfecting typically can be eliminated.

In other embodiments of the invention, a groove, slot or similar opening or indentation may be provided on the inner surface of port 310, and/or the sloped elements 320 may be configured as any other type of upwardly biased resilient element that bends inwardly when insertion element 302 initially is inserted into speculum 300 and then snaps into the provided opening or indentation in port 310, thereby further securing insertion element 302 into speculum 300.

As further shown in FIG. 15, the very distal end 322 of insertion member 318, particularly when configured as a solid optically transmissive light-guiding element, preferably is angled so as to bend the light emerging from member 318 toward the center of the speculum. As a result, the emerging light is directed to where it is most needed by the physician.

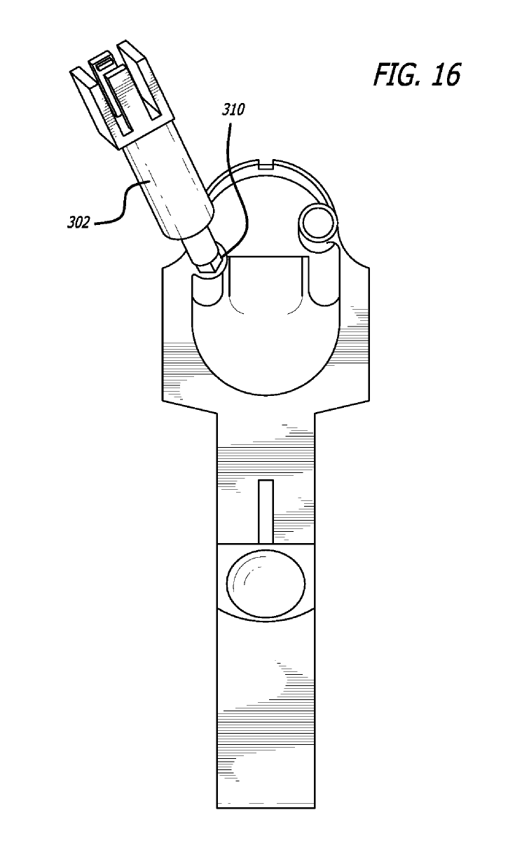

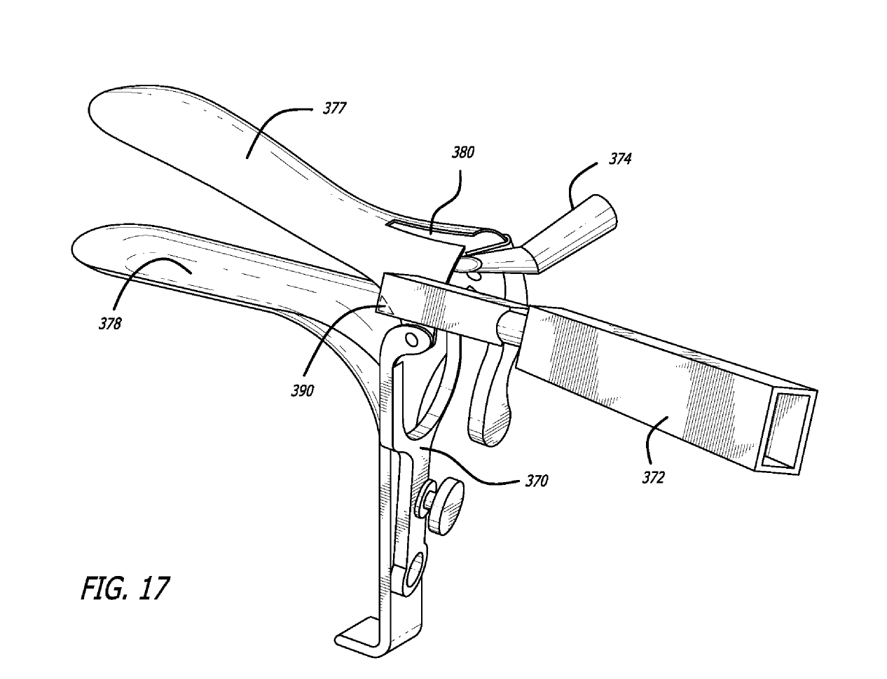

In the present embodiment, as shown in FIG. 16, insertion device 302 preferably is inserted into port 310 at an angle (e.g., up to 45 degrees from vertical), in order to keep both it and the surgical instrument that attaches to it out of the physician's way. The elbow bend 319 (which preferably is up to 45 degrees) helps direct the light into the correct direction and the slanted cut at the distal end of insertion member 318 further directs the light toward the center of the speculum 300.

Attachment Device with Corner Notch

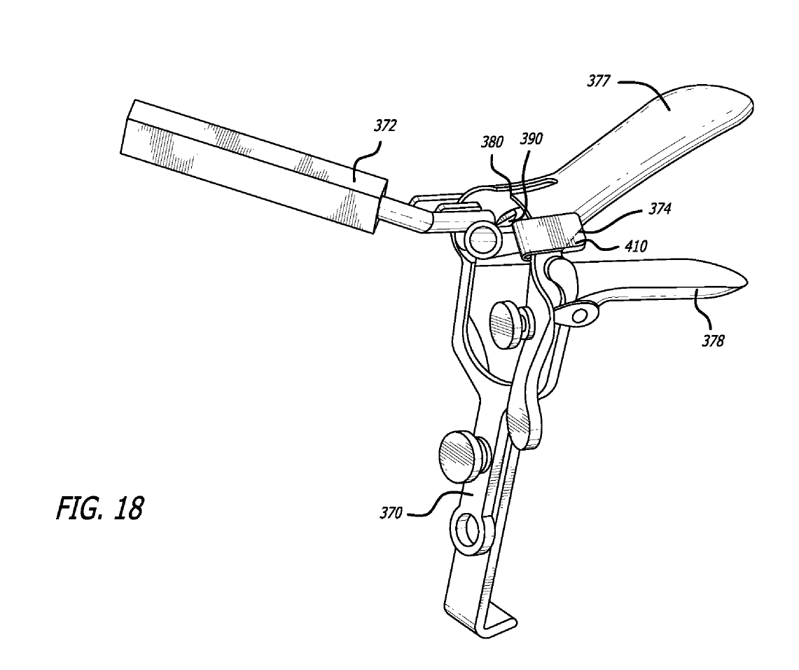

FIGS. 17 and 18 show a speculum 370 and certain attachment devices 372 and 374. Generally, speculum 370 will be made entirely from steel, but instead may be made from any other material or combination of materials. As shown, speculum 370 includes an upper blade 377 and a lower blade 378. In addition, upper blade 377 is provided with one or more slots 380 which may be used for attachment of surgical devices.

Preferably, both of attachment devices 372 and 374 are formed entirely of plastic (e.g., injection molded as a single piece), but instead may be made of any other material or combination of materials. As described in more detail below, each also includes a notch (e.g., disposed on an outer corner of the device) that helps to secure the attachment device onto speculum 370 by engaging with the outer edge of upper blade 377. More specifically, the present inventor has discovered that the weight of a surgical instrument when inserted into (or otherwise attached to) the attachment device 372 or 374 typically will tend to cause attachment device 372 or 374 to rotate backwards relative to speculum 370 and, eventually, to detach from speculum 370. However, the engagement of such a notch against the outer edge of upper blade 377 prevents such rotation, keeping the subject attachment device 372 or 374 in the desired position.

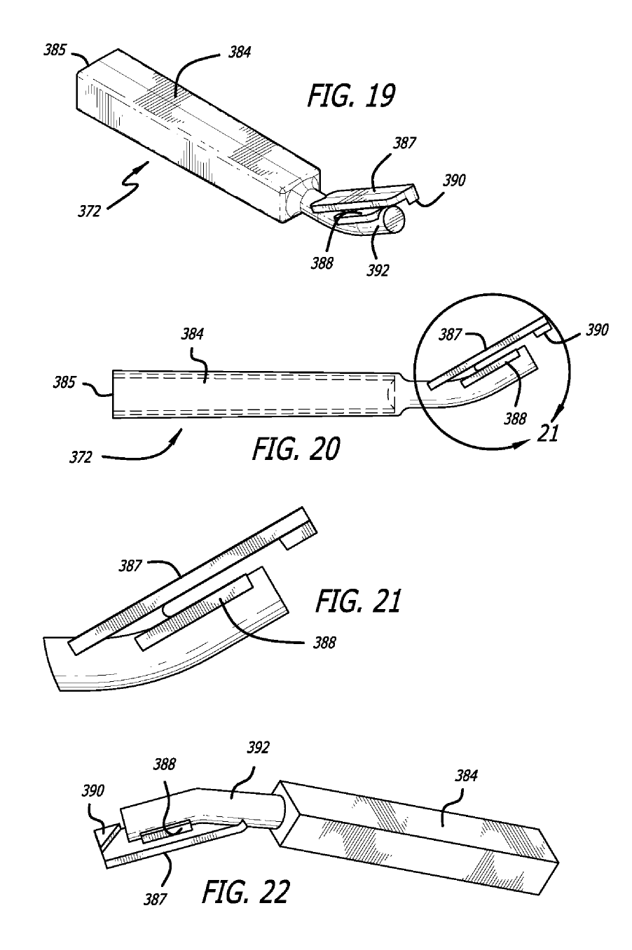

More detail regarding the preferred construction of attachment device 372 is shown in FIG. 19. In this regard, attachment device 372 preferably primarily is used for attachment of a light source to speculum 370 and for guiding the light provided by such light source into a desired direction. Accordingly, attachment device 372 is provided with an attachment apparatus 384 for receiving and/or attaching to such a light source. Once again, a typical light source for use in connection with attachment device 372 is the vaginal illuminator system No. 78103 manufactured and sold by Welch Allyn of Skaneateles Falls, N.Y. In the present embodiment, attachment apparatus 384 is configured as a hollow tube having a rectangular cross-section. As a result, the light source need only be inserted into the open end 385 of attachment device 372. In addition, or instead, attachment apparatus 384 may be provided with a snap-fit mechanism, may be threaded, or may be provided with any other attachment mechanism.

The distal end of attachment device 372 is primarily for attaching to the upper blade 377 of speculum 370. Accordingly, the distal end is provided with two surfaces 387 and 388 that are capable of sandwiching upper blade 377. However, even if a fairly tight fit can be provided by surfaces 387 and 388, attachment device 372 frequently will become detached from speculum 370. In order to prevent this, one of such surfaces (here, upper surface 387) is provided with a notch 390 that extends inwardly (here, downwardly) in order to engage the outer edge of upper speculum blade 377. Such a notch often can act to lock attachment device 372 to speculum 370, particularly when the weight of the surgical device that is inserted into (or otherwise attached to) attachment device 372 tends to rotate attachment device 372 in such a manner as to press notch 398 against the outer edge of blade 377.

In the present embodiment, surfaces 387 and 388 are flat and are approximately parallel to each other, as shown in FIGS. 20 and 21. However, this configuration is not strictly necessary. Instead, surfaces 387 and 388 may be curved (e.g., so as to match the curve of speculum blade 377) and/or configured in any other manner that is appropriate for gripping blade 377.

A guiding member 392 is attached to and disposed beneath lower surface 388 and is used for guiding light in the present embodiment of the invention. More preferably, guiding member 392 is configured as a bent cylinder comprised of clear solid plastic. However, guiding member 392 instead may be configured for guiding a surgical instrument, air and/or fluids, and may be constructed in any other desired manner, preferably matched to its intended purpose. For example, when guiding a surgical instrument, air and/or fluids, guiding element 392 may be configured as a hollow tube.

Notch 390 according to the preferred embodiments of the invention is seen more clearly in FIG. 22. As shown, notch 390 is at least roughly triangular in shape and extends downwardly at the outer corner of upper surface 387. Such a configuration permits attachment device 372 to be angled relative to the outer edge of speculum upper blade 377. Other configurations may be used for different desired positions of attachment device 372.

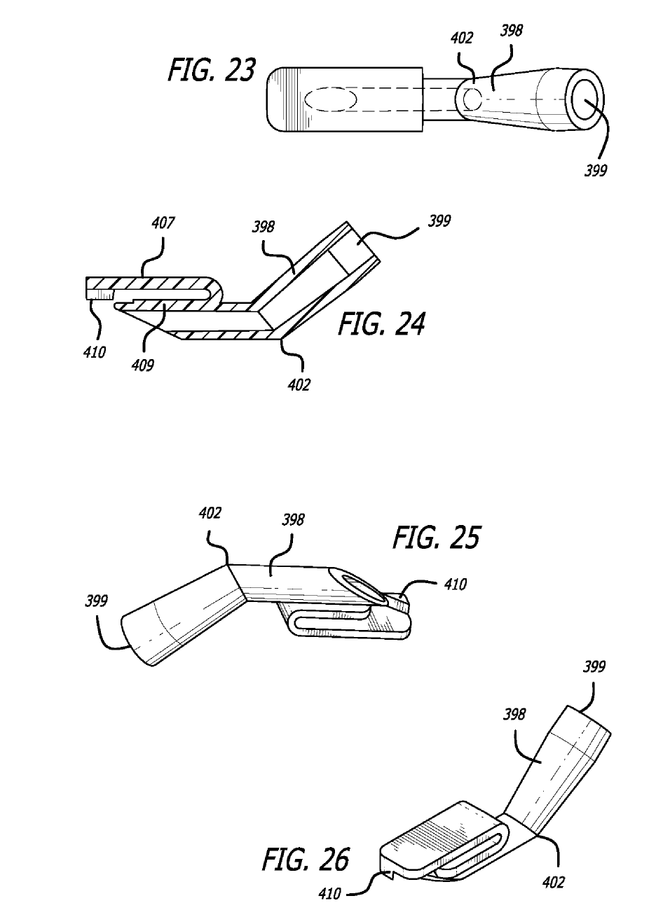

Attachment device 374 (shown in FIGS. 23-26) is similar to device 372, except that is provided with a guiding member 398 for guiding air and/or fluids and is configured for attaching to the right side of speculum 370, rather than the left (as is attachment device 372). Accordingly, a suctioning tube may be fitted around the outside of the distal end 399 of the guiding member 398. Basically, guiding member 398 functions as an extension of a suctioning tube, preferably is more rigid than an ordinary suctioning tube and has an elbow bend 402 to accommodate the angle at which the suctioning tube preferably attaches to speculum 370.

Similar to attachment device 372, device 374 has an upper surface 407 and a lower surface 409 that together are capable of sandwiching the upper blade 377 of the speculum 370. Also, the outer distal corner of upper surface 407 is provided with an inwardly extending notch 410 for engaging with the outer edge of upper blade 377.

In the present embodiment, the light-guiding attachment apparatus 372 is configured for attaching to the left side of speculum 370 while the air/fluid-guiding attachment apparatus 374 is configured for attaching to the right side of speculum 370. While this arrangement of the attached surgical devices is preferred, it is not critical.

Attachment Device With Hooks Having Different Heights

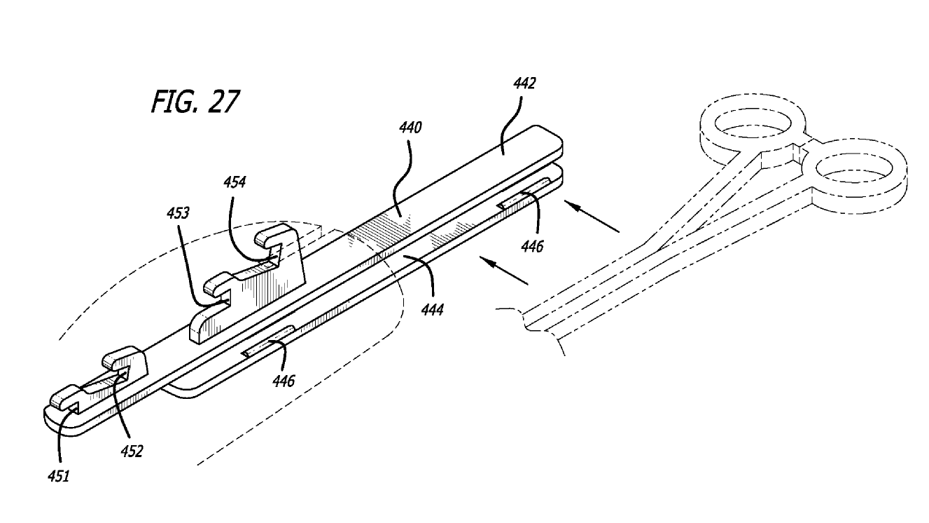

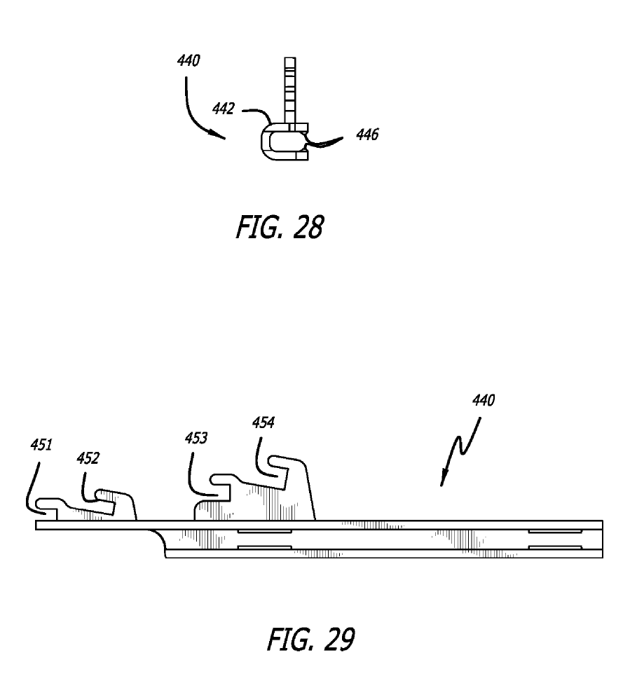

FIG. 27 illustrates a further variation on an attachment apparatus 440 for attaching to a speculum, e.g., to speculum 300 or speculum 370. As shown in FIG. 27, apparatus 440 is provided with a holding structure 442 that is open along one edge 444 for receiving a surgical clamp, tenaculum, retractor or similar medical instrument. In addition, small stabilizing rails 446 preferably are provided to help secure such medical instrument within attachment apparatus 440. As shown in FIG. 28, stabilizing rails 446 preferably are at least approximately triangular in lateral cross-section (for facilitating the insertion and removal of a medical instrument), and holding structure 442 preferably is “C” shaped, having the opening 444 along one of its side edges.

In the preferred embodiments of the invention, the entire apparatus 440 is fabricated from plastic (e.g., as a single injection-molded piece). As a result, apparatus 440 preferably is somewhat pliable, allowing the opening 444 to be temporarily widened as the surgical instrument is inserted therein, but then closing once the surgical instrument clears stabilizing rails 446. Thus, the surgical instrument is securely held in place. More precisely, where a retractor, tenaculum or surgical clamp is being held by attachment apparatus 440, typically only one of the legs of such instrument will be secured to attachment apparatus 440.

Disposed on top of holding structure 442 is a sequence of hooks 451-454 extending along the length of structure 442, e.g., for attaching to the upper blade of a speculum. More preferably, the subject speculum preferably is provided with a slot, such as slot 306 in speculum 300 or slot 380 in speculum 370, and any one of the hooks 451-454 can attach to the speculum at the interior end of such slot. For example, FIG. 27 shows hook 454 attaching to a speculum in this manner. It is noted that the width of the hooks is selected to match (e.g., be slightly wider than) the width of the slot in the speculum. Although four hooks are used in the present embodiment, any other number of hooks instead may be used.

It will be readily appreciated that attachment apparatus 440 is similar to the specific embodiment of the attachment apparatus that is disclosed in the '450 application. For example, each uses a row of hooks that extends along the length of the apparatus. However, there are several differences between the two embodiments. For example, attachment apparatus 440 uses hooks 451-454 having different heights. This feature often can provide the physician with additional flexibility in positioning the surgical or medical instrument that is secured by attachment apparatus 440.

As shown in FIG. 29, the hooks 451-454 are clustered in groups of two along the length of apparatus 440, with the height differences between the adjacent groups being significantly greater than the height differences between adjacent hooks in the same group, and with the heights of the hooks increasing toward the proximal end of the apparatus. Also, the angle of the hook increases from the distal to the proximal end within each group. This configuration has been found to provide the best results, but is not critical to the design.

Additional Considerations

Several different embodiments of the present invention are described above, with each such embodiment described as including certain features. However, it is intended that the features described in connection with the discussion of any single embodiment are not limited to that embodiment but may be included and/or arranged in various combinations in any of the other embodiments as well, as will be understood by those skilled in the art.

Similarly, in the discussion above, functionality may be ascribed to a particular module or component. However, unless any particular functionality is described above as being critical to the referenced module or component, functionality may be redistributed as desired among any different modules or components, in some cases completely obviating the need for a particular component or module and/or requiring the addition of new components or modules. The precise distribution of functionality preferably is made according to known engineering tradeoffs, with reference to the specific embodiment of the invention, as will be understood by those skilled in the art.

Thus, although the present invention has been described in detail with regard to the exemplary embodiments thereof and accompanying drawings, it should be apparent to those skilled in the art that various adaptations and modifications of the present invention may be accomplished without departing from the spirit and the scope of the invention. Accordingly, the invention is not limited to the precise embodiments shown in the drawings and described above. Rather, it is intended that all such variations not departing from the spirit of the invention be considered as within the scope thereof as limited solely by the claims appended hereto.

1. An apparatus for insertion into a port in a speculum, comprising:
(a) a male insertion member that:
(i) is elongated, having a proximal end and a distal end,
(ii) is sized to fit within a port in a speculum, and
(iii) has at least one sloped element on its outer surface which slopes upwardly from the distal end of the male insertion member toward its proximal end; and
(b) attachment means coupled to the male insertion member for at least one of accepting or attaching to a surgical tool,
wherein the sloped element creates a secure fit with the inner surface of the port when the male insertion member is inserted into the port.

2. An apparatus according to [claim 1], wherein the attachment means comprises a tube-like structure for inserting a surgical tool.

3. An apparatus according to [claim 1], wherein the male insertion member functions as a guide member for guiding at least one of: light, airflow, fluid or a surgical instrument.

4. An apparatus according to [claim 3], wherein the guide member is for guiding light.

5. An apparatus according to [claim 4], wherein a distal end of the guide member from which the light emerges is cut at an angle so as to bend the light toward the center of the speculum.

6. An apparatus according to [claim 3], wherein the guide member comprises a hollow tube.

7. An apparatus according to [claim 1], wherein the male insertion member is comprised of plastic.

8. An apparatus according to [claim 1], wherein the male insertion member functions as a guiding means for guiding at least one of: light, airflow, fluid or a surgical instrument.

9. An apparatus according to [claim 8], wherein the guiding means is for guiding light.

10. An apparatus according to [claim 9], wherein a distal end of the guiding means from which the light emerges is cut at an angle so as to bend the light toward the center of the speculum.

11. An apparatus according to [claim 8], wherein the guiding means comprises a hollow tube.

12. An apparatus according to [claim 1], wherein the male insertion member has a non-circular cross section that matches the non-circular cross section of the port of the speculum, thereby preventing inadvertent rotation of the male insertion member in the port.

13. An apparatus according to [claim 1], wherein the male insertion member has at least two sloped elements on its outer surface, each sloping upwardly from the distal end of the male insertion member toward its proximal end.

14. An apparatus according to [claim 13], wherein the at least two sloped elements are distributed at least approximately uniformly around the circumference of the male insertion member.

15. An apparatus according to [claim 1], wherein the at least one sloped element slopes significantly more gradually from the distal end of the male insertion member than from its proximal end.

16. An apparatus according to [claim 1], wherein the secure fit created by the at least one sloped element is a friction fit.

17. An apparatus according to [claim 1], wherein the secure fit created by the at least one sloped element is a snap fit.
